SEQUENCE LISTING (36 sequences)

SEQ ID NO: 1 - DNA, 20 bp, Artificial Sequence
  forward primer
gcttccttagatggcattca

SEQ ID NO: 2 - DNA, 22 bp, Artificial Sequence
  reverse primer
cttccaagaatcagaagtctcc

SEQ ID NO: 3 - DNA, 18 bp, Artificial Sequence
  forward primer
ttgggtggggacacagag

SEQ ID NO: 4 - DNA, 20 bp, Artificial Sequence
  reverse primer
cctggctcaaggaattacaa

SEQ ID NO: 5 - DNA, 20 bp, Artificial Sequence
  forward primer
cgagcacacctacaaaagct

SEQ ID NO: 6 - DNA, 20 bp, Artificial Sequence
  reverse primer
tcgattaggttcagttccca

SEQ ID NO: 7 - DNA, 20 bp, Artificial Sequence
  forward primer
gagcaatgatctcatttgca

SEQ ID NO: 8 - DNA, 25 bp, Artificial Sequence
  reverse primer
aagtaaacatgtataggaaaaagct

SEQ ID NO: 9 - DNA, 20 bp, Artificial Sequence
  forward primer
gaaatgtgctttgacaggaa

SEQ ID NO: 10 - DNA, 22 bp, Artificial Sequence
  reverse primer
caaaaagggacatatgctactt

SEQ ID NO: 11 - DNA, 20 bp, Artificial Sequence
  forward primer
cacaggaggtttgacctgtt

SEQ ID NO: 12 - DNA, 20 bp, Artificial Sequence
  reverse primer
aactgagatggtgccactga

SEQ ID NO: 13 - DNA, 22 bp, Artificial Sequence
  forward primer
agcccattttcataataaatcc

SEQ ID NO: 14 - DNA, 24 bp, Artificial Sequence
  reverse primer
aatcagtgctttctgtactattgg

SEQ ID NO: 15 - DNA, 24 bp, Artificial Sequence
  forward primer
ttggtacttaataaaccctctttt

SEQ ID NO: 16 - DNA, 22 bp, Artificial Sequence
  reverse primer
ctagagggacagaaccaatagg

SEQ ID NO: 17 - DNA, 20 bp, Artificial Sequence
  forward primer
aggggagaaggctagaatga

SEQ ID NO: 18 - DNA, 20 bp, Artificial Sequence
  reverse primer
cagctgacagagcacagaga

SEQ ID NO: 19 - DNA, 20 bp, Artificial Sequence
  forward primer
ttttccagaagctcagatcc

SEQ ID NO: 20 - DNA, 20 bp, Artificial Sequence
  reverse primer
tatccatccatccatcaacc

SEQ ID NO: 21 - DNA, 20 bp, Artificial Sequence
  forward primer
agccagaaggaggaggatta

SEQ ID NO: 22 - DNA, 23 bp, Artificial Sequence
  reverse primer
atctttctgtacttgcagtgagg

SEQ ID NO: 23 - DNA, 24 bp, Artificial Sequence
  forward primer
tatgtatcatcaatcatctatccg

SEQ ID NO: 24 - DNA, 25 bp, Artificial Sequence
  reverse primer
ttaaaatcattttcactgtgtatgc

SEQ ID NO: 25 - DNA, 20 bp, Artificial Sequence
  forward primer
ctgtggtgtaaggttcgctt

SEQ ID NO: 26 - DNA, 20 bp, Artificial Sequence
  reverse primer
tagacttgacctcatgggct

SEQ ID NO: 27 - DNA, 20 bp, Artificial Sequence
  forward primer
agagcccatggagctatctt

SEQ ID NO: 28 - DNA, 20 bp, Artificial Sequence
  reverse primer
ctcagttcaaggaaatggga

SEQ ID NO: 29 - DNA, 19 bp, Artificial Sequence
  forward primer
gtaggatgcagtaggcctc

SEQ ID NO: 30 - DNA, 20 bp, Artificial Sequence
  reverse primer
cctctccttaatcctcagcc

SEQ ID NO: 31 - DNA, 22 bp, Artificial Sequence
  forward primer
tgtctgctaatgaatgatttgg

SEQ ID NO: 32 - DNA, 20 bp, Artificial Sequence
  reverse primer
ccatcccctaaacctctcat

SEQ ID NO: 33 - DNA, 20 bp, Artificial Sequence
  forward primer
cccagatattttgaccacca

SEQ ID NO: 34 - DNA, 20 bp, Artificial Sequence
  reverse primer
ggcatgtggttgctataacc

SEQ ID NO: 35 - DNA, 20 bp, Artificial Sequence
  forward primer
ttccctctctccctctgtct

SEQ ID NO: 36 - DNA, 20 bp, Artificial Sequence
  reverse primer
acacacacccaaaaccagat

(19) United States
(12) Patent Application Publication
(10) Pub. No.: US 2005/0112569 A1
(43) Pub. Date: May 26, 2005

(54) Method of determining blood-relationship by typing str alleles on the x chromosome and dna typing kit using the same

(76) Inventors: Yeon Bo Chung, Seoul (KR); Eun-Young Kim, Seoul (KR)

Correspondence Address: KLARQUIST SPARKMAN, LLP, 121 SW SALMON STREET, SUITE 1600, PORTLAND, OR, 97204, US

(21) Appl. No.: 10/481,951

(22) PCT Filed: Aug. 6, 2002
(86) PCT No.: PCT/KR02/01490

(51) Int. Cl. C12Q 1/68

(52) U.S. Cl. 435/6

(57) ABSTRACT

The present invention relates to a method for determining blood-relationship by typing STR alleles on the X chromosome and a DNA typing kit using the same, particularly when the father's DNA is not present. According to the method of the present invention, the blood-relationship of a suspected-grandmother and a suspected-granddaughter can be confirmed in the absence of the father's DNA by comparing STR alleles on the X chromosome of each subject. As for step-sisters born of different mothers, their blood-relationship can be determined by investigating whether they share STR alleles on the X chromosome when their mothers' alleles on the X chromosome were excluded. Therefore, the method of the present invention can be usefully used in determining the blood-relationship in the absence of the father's DNA.

### FIELD OF THE INVENTION

The present invention relates to a method for determining blood-relationship by typing short tandem repeat (STR) DNA on the X chromosome and a DNA typing kit using the same. More precisely, the present invention relates to a method for confirming blood-relationship by comparing STR DNA on the X chromosome particularly when the father's DNA is not present and a DNA typing kit using thereof.

### BACKGROUND ART OF THE INVENTION

It has been known that only about 4% of human DNA is encoding functional genetic information and the rest of them have no such function. In case of DNA without genetic information, a certain base sequence is repeatedly shown or the same base sequence is given here and there on the genome. When the certain base sequence is repeated within a region, it is called as “tandem repeat”. The non-informative DNA does not affect on cell function even when base sequence is changed by errors during the replication process or by recombination since the DNA basically does not have any genetic information. Thus, it can be said that the selection pressure of the DNA is “zero” and accordingly lots of accumulated changes in base sequences can be found in them. Especially in case of tandem repeated base sequences, the repeating numbers of basic base sequence are vary, which is proven by the comparison test of the same region of DNA extracted from various people.

The variation of tandem repeated base sequence was first found by Professor White at University of Utah (USA) in 1980 and later forensically applied by Professor Jeffries at University of Leister (England) in 1985. Especially, it has been applied from then on to identify a person using DNA, resulting in the birth of the term “DNA fingerprint”. The variation of repeating times is not serious and even limited in a tandem repeat genetic locus. However, when the variations happening in tandem repeat genetic loci of various chromosomes are considered together, lots of peculiar combinations can be made. For example, a coin has only two sides: front and back. When it is thrown 10 times, the chance of getting n times front or the chance of getting m times back is very few. But once chances are combined and permutation is made, for example the chance of front in first throw and back in second throw, etc, the peculiarity is much increased. The certain combination of tandem repeated alleles is called “DNA profile” and the DNA profile consisting of 10 or more combinations of tandem repeated genetic loci is unique enough to identify each person on earth.

The variation of tandem repeat is huge but the variation gap between generations is small as long as mutation is the cause. For example, there is not much difference of repeating times of tandem repeated base sequence between DNAs of father and son. Owing to this characteristic, tandem repeated DNA could be effectively used for the determination of blood-relationship. It is now generalized throughout 80 or more countries to decide whether an alleged father is real biological father by comparing DNA profiles and to identify a suspect by comparing his DNA with the sample DNA taken from crime scene.

Tandem repeated allele was analyzed by Southern blot technology at the early days. Precisely, genomic DNA was digested with a certain restriction enzyme such as HaeIII and made into fragments, followed by electrophoresis. Then, probe having base sequence that is hybridized with tandem repeated base sequence and marked with isotopes was used for the reaction with the above DNA fragments, resulting in the presence of tandem repeated DNA fragments on the film. Fragments having same size were regarded as same alleles. In order to perform this method, comparatively large amount of specimen is required in addition to the tiresome process. Thus, this method is now limited in use and polymerase chain reaction (PCR) is replacing it. In the late 1980s, many STR base sequences that can be easily and precisely amplified by PCR were found, and therefore, more precise allele typing became possible. Further studies and tests have been undergoing in the United States and FBI had selected a standard test with 13 STRs and officially announced it as a standard for a genetic identification.

Exact typing of STR is very difficult without denaturing acrylamide gel electrophoresis that has been used to decide base sequence since STR is generally no more than hundreds of base pair (bp) long. Denaturing acrylamide gel electrophoresis is very troublesome test method that requires complicated treatment procedures like silver staining or marking with radioisotopes to show the fragments. In order to make this difficult experiment easy and simple, automatic gel electrophoresis system or automatic sequencer was developed, with which fluorescence marking was enabled and the test results were shown rightly. Applied Biosystems, an American company, developed an automatic electrophoresis system such as ABI310 possibly using 4 different fluorescent dyes, and Promega brought out a primer kit enabling up to 15 STRs to be typed simultaneously by electrophoresis after marking each bundle of STRs tied by 3-4 different sized STRs with different fluorescent dyes.

Though STR is very useful for identification or paternity test, it is not very helpful when the alleged relationship is far from father and son or the amount of sample is small. For example, it is difficult to determine the cousin-relationship by STR and to identify someone when only a hair fragment without its root is given.

Although mitochondria DNA typing is limited in use for the cases of a matrilineal heredity, it has been rapidly developed together with STR to identify a biological mother or to analyze the infinitesimal quantity of sample. A cell has hundreds to tens of thousands of mitochondria and each mitochondrion has tens of thousands or hundreds of thousands of DNA. Therefore, the mitochondria DNA outnumber nuclear DNA, so that mitochondria DNA could be an easy target for genetic analysis. About 17 kb-long mitochondria DNA is mostly encoding proteins or tRNA and has comparatively small variation. However, a certain locus that is 1 kb-long and is coding various regulation signals shows a big difference among people, from which it can be possibly confirmed whether the subjects have same maternal line or even further identified whether the subjects are derived from the same person.

While the maternal line can be confirmed by analyzing mitochondria DNA, the paternal line can be determined by typing STR on Y chromosome along with other STR. Except a short end, Y chromosome is transmitted to the next generation without recombination, meaning that every male descendants share the same Y chromosome except both ends if they have same ancestor. Among many disclosed STRs from Y chromosome, about 10 STRs that have comparatively more alleles and can be easily typed have been selected and used. STRs of Y chromosome have been importantly used in finding male traces from a raped body and also can be an important clue to decide whether descendents share the same ancestor even after several generations.

Owing to their unique characteristics as shown above, mitochondria DNA and STR of Y chromosome became important tools for the identification or determination of blood-relationship even for the cases that cannot be confirmed by STR of autosome and are also very useful for supplementing STR of autosome. With all these methods, it is still difficult to determine a distant relation over farther and son and hardly can get a result from a test of real child without farther. Just in case that brothers or parents of the deceased father are alive, the relationship could be only indirectly presumed on the assumption that the deceased father surely had blood ties with them. If so, accurate results cannot be guaranteed in most cases.

Thus, in order to solve the above problems, the present inventors have studied and established the way to confirm the relationship of father and daughter by comparing STR of X chromosome in the absence of father's DNA. And the present invention has accomplished by finding certain STRs of X chromosome that are useful for the identification and proving that the application thereof is successful.

### SUMMARY OF THE INVENTION

It is an object of the present invention to provide a method to confirm blood-relationship between a suspected-grandmother and a suspected-granddaughter by comparing STR alleles on the X chromosome of each subject in the absence of the father's DNA and/or to determine blood-relationship between sisters by investigating whether they share STR alleles on the X chromosome when their mothers' alleles on the X chromosome were excluded.

### BRIEF DESCRIPTION OF THE DRAWINGS

FIG. 1a is a pedigree showing a typical family line required for the blood-relationship test by comparing STR alleles on the X chromosomes of grandmother and granddaughter,

FIG. 1b is a pedigree showing a typical family line required for the blood-relationship test by comparing STR alleles on the X chromosomes of sisters,

FIG. 3 is a diagram showing how X chromosome is transmitted from grandparents to grandchildren and how it is used for the determination of blood-relationship,

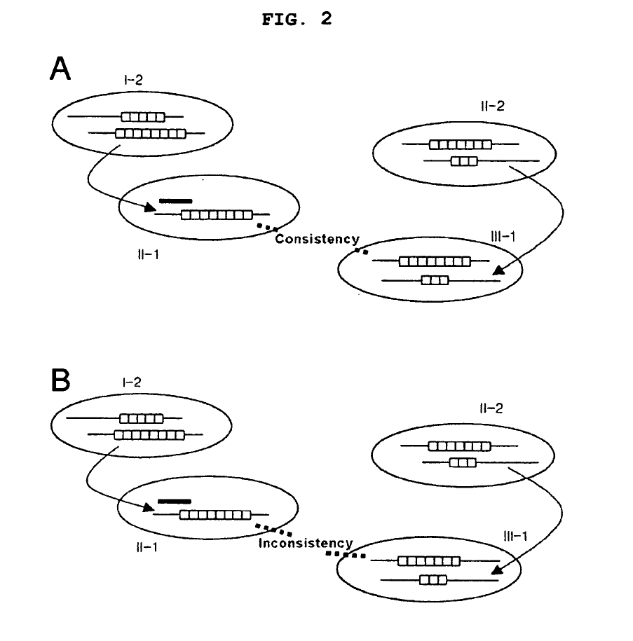

A: The case of real children,

B: The case of non-real children

FIG. 3 is a diagram showing the location of selected 18 STRs on X chromosome,

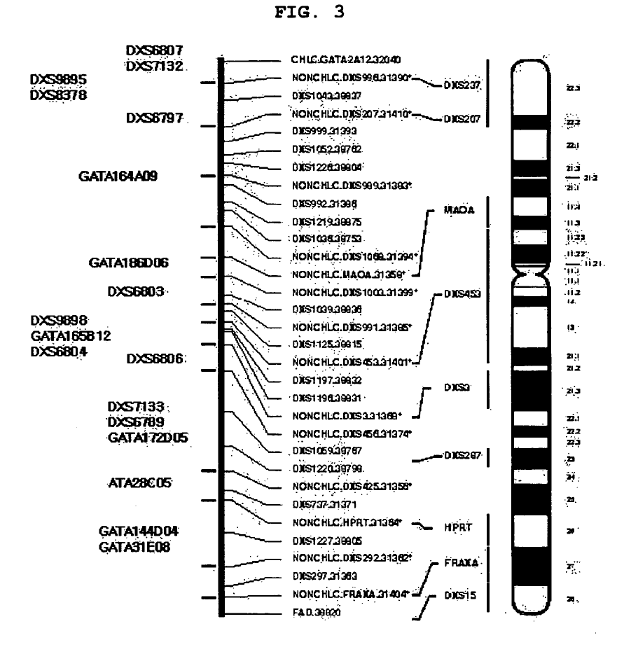

FIG. 4 is a diagram showing the steps of 18 STR alleles on X chromosome.

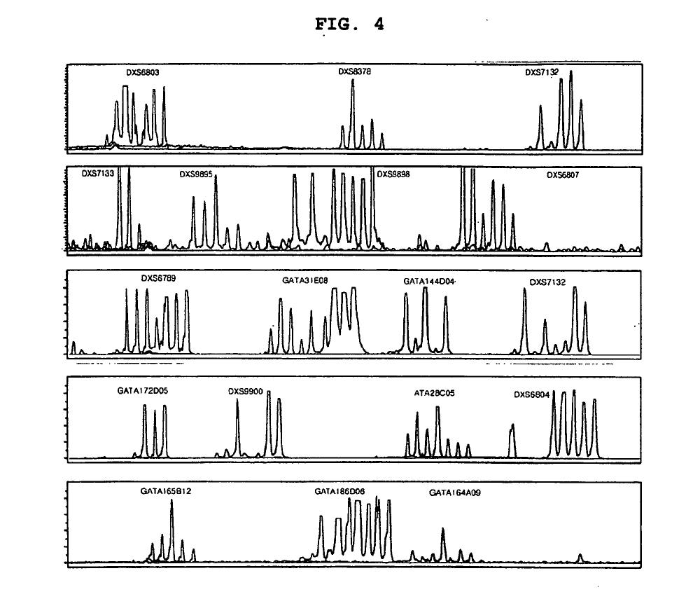

### DETAILED DESCRIPTION OF THE INVENTION

To achieve the above objects, the present invention provides 18 STR alleles on X chromosome and a method to apply thereof for the determination of blood-relationship between a suspected-grandmother and a suspected-granddaughter or between suspected-sisters.

The present invention also provides a DNA typing kit, with which the above STR alleles on X chromosome are effectively used.

Hereinafter, the present invention is described in detail.

The present invention provides 18 STR alleles on X chromosome and a method to apply thereof for the determination of blood-relationship between a suspected-grandmother and a suspected-granddaughter or between suspected-sisters.

Particularly, the present invention provides a method for investigating STR alleles on X chromosome of a women to see if there is a STR on X chromosome transmitted from father when their mother's alleles on X chromosome are excluded based on the fact that a man has only one X chromosome and a father's X chromosome is transmitted to a daughter as it is, and for determining blood-relationship by comparing STR alleles on X chromosome of subjects in the absence of father's DNA.

The method for determining blood-relationship comprises the following steps:
1) extracting DNA from specimens of examinee;
2) obtaining massive STR DNA through PCR performed with STR on X chromosome using the DNA obtained in the above step 1 as a template;
3) investigating the physical, chemical characteristics of STR DNA obtained in the above step 2;
4) determining blood-relationship by comparing types of STR obtained in the above step 3.

In the above step 1, one or more components can be selected as specimens from a group consisting of blood, hair, saliva, epidermis, sperm, samples extracted from vagina, separated cells, tissue samples, dandruffs, ashes, etc, and mixed.

For the above step 2, it is preferable to select STR that has high rate of heteroconjugation on X chromosome and lots of alleles for better identification and to use standard allele steps obtained from cloning or PCR with whole alleles as molecular weight markers. Preferably selected STRs are DXS7133, DXS9895, DXS9898, DXS6807, DXS6803, DXS8378, DXS7132, DXS6789, GATA31E08, DXS9900, GATA144D04, GATA165B12, GATA186D06, GATA164A09, DXS6806, ATA28C05, DXS6804 or DXS6797.

It is also preferable to use multiplex PCR that amplifies simultaneously 3 to 10 different sized STRs as a whole for simultaneous PCR and analysis of many STRs. At this time, it is required to arrange primer sequences for the amplification of each STR not to disturb each other. Since the distinguishment of alleles by electrophoresis depends on the size of fragments, the size of STR alleles for electrophoresis is also required not to be overlapped. Considering the limited fragment separating capacity of electrophoresis, 3-4 different sized STRs are supposed to be filed for a one panel and marked with radioisotope or fluorescence.

The way of marking is to attach fluorescent material or radioisotope to the 5′ end of a primer or to the 3′ end by taking advantage, of the reaction of terminal deoxynucleotidyl transferase after PCR. The other way to confirm fragments without marking is to perform silver staining after electrophoresis. It is possible to mark various multiplexing panels with all different colored fluorescent materials owing to their various wavelengths. It is also possible to trace fragments marked by various fluorescent materials using automatic electrophoresis system such as ABI310 provided by Applied Biosystems.

Each STR alleles show different repeating times, resulting in the difference in length of amplified fragments. Thus STR alleles can be identified by measuring their length by electrophoresis. The way to measure the length by electrophoresis is to measure migration distance of a fragment per time unit or to measure time that takes for a fragment to move a certain distance when automatic electrophoresis system is used. The migration induced by electrophoresis is influenced not only by the length of a fragment but also by the structure or sequence of the fragment, so that standard alleles should be used as a criterion for comparison in order to determine correct alleles. Therefore, using allele steps is essential for genetic identification by STR. The present invention provides the allele steps of 18 STRs.

The above step 3 is for the analysis of physical and chemical characteristics of the extracted DNA. Precisely, this stage includes the procedure of electrophoresis that measures the length of DNA fragments amplified by PCR, method to use mass spectrometer to measure the mass of the amplified fragments, method to measure the differences among base sequences caused by hybridization and method to determine the base sequence directly. In order to investigate hybridization, it is included to use DNA array representing the characteristics of sample DNA by attaching the established standard DNA whose properties are already disclosed like DNA chip to the surface of a matrix for further reaction with the sample DNA.

In order to investigate physical and chemical characteristics of DNA in the above step 3, a certain device that is able to perceive a mark attached to sample DNA is necessary, otherwise DNA fragments should be treated to be detected well. Autoradiography and scintillation counting methods are preferably used to detect DNA marked with radioisotopes, ABI automatic sequencer provided by Applied Biosystems or FMBIO provided by Hitachi is useful for detecting DNA marked with fluorescent materials, and silver staining method is suitable for detecting unmarked DNA.

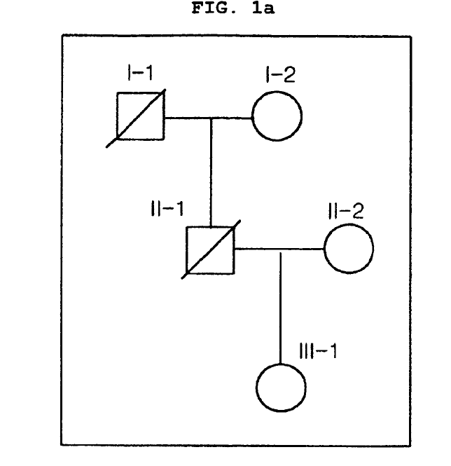

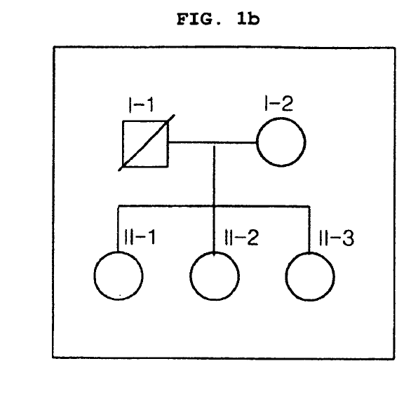

The method for determining blood-relationship of the present invention is based on the fact that a man has only one X chromosome that is transmitted to his daughter directly. Thus, when X-STR that is shared with her mother is excluded, the remaining X-STR of daughter is shared with her father, and the father's X-STR is transmitted from grandmother. Repeatedly speaking, one of the two X chromosomes of grandmother is transmitted to father and then it is transmitted to daughter as it is. Therefore, grandmother and granddaughter share half of X-STR each other if they are in a lineal relation. FIG. 1a is a diagram showing the transmission mode of X chromosome. In this diagram, a rectangle represents a man and a circle represents a woman. An oblique line on a rectangle means he is deceased. Each generation is represented by the Roman numbers I, II, III, etc, and each individual of each generation is represented by the Arabic numbers. Father(II-1)'s X chromosome is not exactly same as one of the two X chromosomes of grandmother(I-2) since STR alleles of grandmother are exchanged each other by the recombination of her two X chromosomes during the meiosis. However, half of granddaughter(III-1)'s STR alleles on X chromosome are exactly same as those of grandmother in case they are in a direct line.

More precisely, as comparing grandmother(I-2)'s DNA with that of suspected-granddaughter(III-1) as seen in FIG. 2, the consistency of 8 repetition on a specific STR suggests they are in a direct line(A), but if the number of repetition is not in accord, it can be determined that they are not in a direct line(B). The black bar in FIG. 2, II-1 represents Y chromosome. If grandmother (I-2) s X chromosome is transmitted to granddaughter(III-1) by way of father(II-1), every STR alleles on father's X chromosome should be reflected in granddaughter's X chromosome. If grandmother and granddaughter are not in a blood relationship, each STR allele has much chance to be different each other and most STR hardly have chance to have consistent alleles continuously. Therefore, blood-relationship can be determined by whether STR alleles are shared by subjects; that is, when most of STR alleles of subjects are repeatedly consistent, it can be determined that they are in a blood-relationship.

The way to confirm a blood-relationship by comparing STR on X chromosome can be applied for determining a blood-relationship between suspected-sisters. FIG. 1b is a pedigree of a family that has 3 daughters. X-STR of a woman is made up of one transmitted from father and the other from mother. Thus, when X-STR alleles transmitted from mother are excluded, the rest alleles must be in accord with X-STR alleles of father. It is all the same to every sister, meaning they share the same X-STR alleles when alleles from mother are excluded if they have a same biological father.

In order to confirm a real child of a deceased father by the method of the present invention, it ought to be investigated whether grandmother's X-STR alleles were transmitted to granddaughter through father. If the subjects are not in a blood-relationship, the chances are very low for them to have same alleles.

Therefore, the present inventors have confirmed whether the X-STR alleles of grandmother were transmitted to granddaughter.

At first, the inventors have randomly selected 18 STRs evenly spread on X chromosome, and analyzed the alleles of those 18 STRs. As a result, at least one of granddaughter's alleles has been found in grandmother's alleles. And granddaughter has been confirmed to have the same alleles as grandmother's alleles when alleles transmitted from mother were excluded, proving that half of grandmother's X-STR alleles have to be transmitted to granddaughter through father (see Table 1).

TABLE 1
STR profile of X chromosomes of grandmother and
granddaughter

| | Size of PCR | | | | | |
|---|---|---|---|---|---|---|
| | Fragment | | | | | |
| | Grand- | | Gene Profile | | | |
| STR | mother | Granddaughter | Grandmother | | Granddaughter | |
| DXS9898 | 201/205 | 201/205 | 6 | 7 | 6 | 7 |
| DXS6807 | 252/264 | 252/264 | 2 | 5 | 2 | 5 |
| DXS6803 | 114 | 114/118 | 2 | 2 | 2 | 3 |
| DXS8378 | 210/214 | 206/214 | 3 | 4 | 2 | 4 |
| DXS7132 | 283/287 | 287 | 3 | 4 | 4 | 4 |
| DXS6789 | 119/136 | 136 | 3 | 7 | 7 | 7 |
| GATA31E08 | 243 | 239/243 | 5 | 5 | 4 | 5 |
| DXS9900 | 122/126 | 122/126 | 4 | 5 | 4 | 5 |
| GATA144D04 | 237 | 237/249 | 3 | 3 | 3 | 6 |
| GATA165B12 | 125/134 | 125/129 | 1 | 3 | 1 | 2 |
| GATA186D06 | 205/209 | 200/208 | 3 | 4 | 2 | 4 |
| GATA164A09 | 246/250 | 250 | 5 | 6 | 6 | 6 |
| DXS6806 | 167/171 | 167 | 2 | 3 | 2 | 2 |
| ATA28C05 | 279/288 | 279/282 | 3 | 6 | 3 | 4 |
| DXS6804 | 187 | 178/187 | 6 | 6 | 4 | 6 |
| DXS6797 | 259 | 259/263 | 2 | 2 | 2 | 3 |

Next, the present inventors have also confirmed that there is almost no chance for subjects to have the same alleles in case they are not in a blood-relationship.

Although alleles are many and various, there is still a chance to have the same alleles on a specific STR even between the two people not in a blood-relationship. Each target STR for genetic identification generally locates on a different chromosome. Thus, the STRs are transmitted to a descendent independently since they are not linked. Generally used STR sets are reported to have reached Hardy-Weinberg Equilibrium and Linkage Equilibrium since they locate on different chromosomes. Therefore, the possibility that STR alleles are accidentally consistent between subjects is calculated by multiplying the frequency of each STR alleles. However, as for X-STR alleles, they locate on a same chromosome and supposed to be in a linkage even though DNA could be changed between homologous chromosomes by recombination. Thus, it is difficult to estimate the possibility of accidental accordance of X-STR alleles by multiplying their frequencies.

The present inventors have obtained the DNA profiles representing the combination of 18 X-STR alleles for 59 Korean women to investigate the possibility that granddaughter's alleles from father can be accidentally included in grandmother's DNA profile (see Table 2 and 3).

TABLE 2
18 X STR profiles of Korean women

| | DXS7133 | | DXS9895 | | DXS9898 | | DXS6807 | | DXS6803 | | DXS8378 | | DXS7132 | | DXS6780 | | GATA31E08 | |
|---|---|---|---|---|---|---|---|---|---|---|---|---|---|---|---|---|---|---|
| 1 | 5 | 5 | 2 | 3 | 6 | 8 | 1 | 1 | 3 | 3 | 2 | 2 | 2 | 5 | 3 | 8 | 3 | 3 |
| 2 | 4 | 5 | 2 | 4 | 5 | 6 | 4 | 3 | 2 | 2 | 2 | 2 | [symbol] | 5 | 2 | 3 | 1 | 5 |
| 3 | 4 | 4 | 2 | 2 | 6 | 7 | 1 | 1 | 2 | 4 | 3 | 4 | 5 | 6 | 8 | 8 | 6 | 5 |
| 4 | 4 | 4 | 1 | 1 | 6 | 6 | 1 | 4 | 3 | 3 | 2 | 4 | 4 | 5 | 7 | 9 | 4 | 3 |
| 5 | 4 | 5 | 1 | 3 | 6 | 7 | 1 | 4 | 3 | 3 | 2 | 3 | 4 | 5 | 3 | 3 | 5 | 5 |
| 6 | 4 | 4 | 1 | 3 | 6 | 7 | 4 | 4 | 2 | 3 | 3 | 3 | 4 | 5 | 8 | 10 | 4 | 5 |
| 7 | 4 | 4 | 1 | 1 | 6 | 7 | 1 | 5 | 2 | 3 | 2 | 3 | 3 | 5 | 7 | 8 | 4 | 6 |
| 8 | 4 | 4 | 1 | 2 | 6 | 7 | 1 | 4 | 2 | 3 | 3 | 4 | 4 | 5 | 3 | 8 | 4 | 5 |
| 9 | 4 | 6 | 2 | 3 | 6 | 6 | 4 | 6 | 2 | 2 | 2 | 4 | 3 | 5 | 3 | 8 | 1 | 5 |
| 10 | 4 | 6 | 2 | 2 | 6 | 7 | 1 | 5 | 3 | 3 | 3 | 4 | 5 | 5 | 2 | 7 | 3 | 3 |
| 11 | 4 | 4 | 1 | 3 | 6 | 8 | 1 | 5 | 2 | 2 | 3 | 3 | 4 | 5 | 7 | 8 | 3 | 5 |
| 12 | 4 | 4 | 2 | 3 | 6 | 8 | 4 | 6 | 2 | 2 | 2 | 4 | 4 | 4 | 2 | 7 | 4 | 5 |
| 13 | 5 | 7 | 2 | 2 | 7 | 7 | 5 | 5 | 3 | 3 | 2 | 2 | 3 | 4 | 2 | 2 | 1 | 3 |
| 14 | 4 | 6 | 2 | 3 | 6 | 6 | 4 | 5 | 2 | 3 | 2 | 3 | 3 | 4 | 3 | 4 | 4 | 5 |
| 15 | 5 | 6 | 2 | 2 | 3 | 6 | 1 | 4 | 2 | 2 | 2 | 4 | 2 | 5 | 2 | 3 | 5 | 5 |
| 16 | 4 | 6 | 1 | 4 | 6 | 7 | 1 | 4 | 2 | 3 | 2 | 3 | 4 | 4 | 2 | 3 | 3 | 3 |
| 17 | 4 | 4 | 1 | 1 | 6 | 7 | 1 | 4 | 3 | 3 | 2 | 2 | 3 | 3 | 7 | 10 | 2 | 5 |
| 18 | 4 | [symbol] | 2 | 2 | 6 | 8 | 1 | 1 | 2 | 2 | 4 | 4 | 3 | 5 | 7 | 9 | 3 | 4 |
| 19 | 4 | 5 | 2 | 2 | 6 | 6 | 1 | 5 | 1 | 3 | 3 | 3 | 2 | 4 | 2 | 8 | 4 | 5 |
| 20 | 4 | 4 | 1 | 1 | 6 | 7 | 3 | 4 | 2 | 2 | 2 | 3 | 4 | 4 | 3 | 8 | 3 | 5 |
| 21 | 4 | 4 | 1 | 1 | 6 | 7 | 4 | 6 | 2 | 2 | 2 | 2 | 3 | [symbol] | 3 | 3 | 4 | 5 |
| 22 | 4 | 4 | 1 | 1 | 6 | 7 | 1 | 4 | 3 | 3 | 2 | 2 | 2 | 3 | 4 | 7 | 1 | 5 |
| 23 | 4 | 4 | 1 | 1 | 7 | 7 | 1 | 1 | 2 | 2 | 3 | 3 | 2 | 3 | 3 | 7 | 3 | 6 |
| 24 | 4 | 4 | 2 | 2 | 5 | 6 | 1 | 4 | 2 | 6 | 2 | 3 | 3 | 5 | 3 | 7 | 3 | 3 |
| 25 | 5 | 5 | 1 | 1 | 6 | 7 | 4 | 5 | 3 | 4 | 2 | 2 | 2 | [symbol] | 3 | 7 | 4 | [symbol] |
| 26 | 4 | 4 | 2 | 2 | 6 | 6 | 1 | 1 | 2 | 2 | 2 | 3 | 4 | 5 | 9 | 9 | 1 | 6 |
| 27 | 4 | 4 | 2 | 2 | [symbol] | 7 | 1 | 4 | 1 | 4 | 2 | 3 | 5 | 5 | 3 | 3 | 5 | [symbol] |
| 28 | 4 | 5 | 1 | 1 | 5 | 6 | 1 | 5 | 2 | 3 | 2 | 4 | 4 | 4 | 2 | 7 | 3 | [symbol] |
| 29 | 4 | 4 | 1 | 1 | 6 | 6 | 1 | 1 | 2 | 2 | 2 | 2 | 3 | 5 | 3 | 8 | 1 | 3 |
| 30 | 4 | 4 | 2 | 2 | 3 | 7 | 1 | 4 | 2 | 3 | 2 | 2 | 4 | 4 | 2 | 2 | 5 | 5 |
| 31 | 4 | 4 | 2 | 2 | 8 | 8 | 1 | [symbol] | 1 | 3 | 2 | 3 | [symbol] | 4 | 8 | 8 | 4 | 4 |
| 32 | 4 | 5 | 1 | 1 | 3 | 7 | 4 | 3 | 3 | 3 | 2 | 2 | 5 | 5 | 8 | 8 | 4 | 4 |
| 33 | 4 | 4 | 2 | 2 | 7 | 8 | 4 | 4 | 2 | 3 | 2 | 2 | 4 | 6 | 6 | 7 | 3 | 5 |
| 34 | 5 | 5 | 1 | 2 | 6 | 7 | 1 | [symbol] | 3 | 3 | 2 | 2 | 3 | [symbol] | 2 | 3 | 4 | 4 |
| 35 | 4 | 4 | 2 | 4 | 6 | 6 | 1 | [symbol] | [symbol] | 3 | 3 | 4 | 3 | 4 | 2 | 2 | 5 | 6 |
| 36 | 4 | [symbol] | 1 | 3 | 7 | 7 | 1 | 1 | 1 | 3 | 2 | 3 | 3 | 4 | 8 | 8 | 5 | 6 |
| 37 | 5 | 6 | 1 | 3 | 6 | 7 | 1 | 4 | 2 | 3 | 2 | 2 | [symbol] | 5 | 8 | 9 | 3 | 4 |
| 38 | 4 | [symbol] | 1 | 2 | 6 | 8 | 4 | 4 | 2 | 2 | 2 | [symbol] | 3 | 3 | 3 | 7 | 1 | 4 |
| 39 | 5 | 6 | 3 | 4 | 6 | 7 | 3 | 4 | 1 | 1 | 2 | [symbol] | [symbol] | [symbol] | 8 | 8 | 6 | 6 |
| 40 | 4 | [symbol] | 1 | 4 | 6 | 7 | 4 | 6 | 2 | 3 | 2 | 3 | 2 | 5 | 2 | 9 | [symbol] | [symbol] |
| 41 | 4 | [symbol] | 1 | 1 | [symbol] | 8 | 1 | 4 | 1 | 4 | 2 | [symbol] | 5 | 6 | 2 | 3 | 4 | [symbol] |
| 42 | 4 | 4 | 1 | [symbol] | 6 | 6 | 1 | 5 | 3 | 4 | 4 | 5 | 2 | 3 | 2 | 8 | 1 | 3 |
| 43 | 4 | [symbol] | 1 | [symbol] | 6 | 7 | 1 | 1 | 3 | 3 | 2 | 2 | 3 | 3 | 3 | 8 | 4 | 4 |
| 44 | [symbol] | 4 | 1 | 2 | 5 | 7 | 1 | 4 | 2 | 3 | 2 | 3 | 2 | 3 | 3 | 3 | 4 | 5 |
| 45 | 4 | 4 | 2 | 4 | 6 | 7 | 4 | 4 | 2 | 3 | 2 | 3 | 6 | 6 | 6 | 6 | 5 | 5 |
| 46 | 5 | [symbol] | 1 | 3 | 7 | 7 | 1 | 4 | 3 | 3 | 2 | 3 | 3 | [symbol] | 3 | 3 | 5 | 6 |
| 47 | 4 | 4 | 2 | 2 | 3 | 9 | 1 | [symbol] | 2 | 4 | 2 | 3 | 3 | 4 | 2 | 2 | 3 | 3 |
| 48 | 4 | [symbol] | 1 | 1 | 6 | 7 | 1 | [symbol] | 2 | 3 | 3 | 3 | 2 | 4 | 3 | 3 | 4 | 4 |
| 49 | 4 | 4 | 1 | 1 | 6 | 8 | 1 | 1 | 2 | 2 | 2 | 3 | 4 | 4 | 3 | 7 | 5 | 6 |
| 50 | 4 | 5 | 1 | 3 | 8 | 8 | 5 | [symbol] | 1 | 2 | 2 | 3 | 4 | 5 | 2 | 8 | 3 | 4 |
| 51 | 4 | 4 | 2 | 4 | [symbol] | 6 | 4 | 5 | 2 | 4 | 2 | 2 | 4 | 3 | [symbol] | 8 | 3 | [symbol] |
| 52 | 4 | 5 | 1 | 5 | 7 | 8 | 5 | 6 | 2 | 3 | 2 | 2 | 6 | 6 | 7 | 8 | [symbol] | 6 |
| 53 | 4 | 4 | 3 | 5 | 6 | 7 | 4 | [symbol] | 3 | 3 | 2 | 3 | 4 | [symbol] | 3 | 7 | 3 | [symbol] |
| 54 | 4 | [symbol] | 1 | 2 | 3 | 7 | 1 | 4 | 2 | 2 | 2 | 2 | 4 | 4 | 7 | 7 | 2 | 2 |
| 55 | 4 | [symbol] | 2 | 4 | 6 | 7 | 1 | 4 | 2 | 3 | 2 | 4 | 3 | 6 | 3 | 3 | [symbol] | 6 |
| 56 | 4 | 4 | 1 | 1 | 6 | 6 | 4 | 4 | 2 | 2 | 2 | 2 | 4 | 4 | 4 | 8 | 3 | [symbol] |
| 57 | 4 | 6 | 3 | 4 | 6 | 7 | 4 | 5 | 2 | 2 | 2 | 2 | 4 | 6 | 8 | 10 | 3 | 3 |
| 58 | 4 | [symbol] | 1 | 3 | 7 | 7 | 1 | 1 | 2 | 4 | 2 | 3 | [symbol] | [symbol] | 7 | [symbol] | 4 | 6 |
| 59 | 5 | [symbol] | [symbol] | 3 | 6 | 6 | 2 | 4 | 3 | 3 | 2 | 3 | 2 | 4 | 2 | 4 | 3 | [symbol] |

TABLE 3
18 X STR profiles of Korean women

| | DXS9900 | | GATA144D04 | | GATA165B12 | | GATA186D06 | | GATA164A09 | | DXS6806 | | ATA28C05 | | DXS6804 | | DXS6797 | |
|---|---|---|---|---|---|---|---|---|---|---|---|---|---|---|---|---|---|---|
| 1 | 5 | 6 | 3 | 4 | 1 | 2 | 3 | 4 | 6 | 6 | 2 | 2 | 2 | 4 | 4 | 7 | 4 | 7 |
| 2 | 5 | 6 | 3 | 5 | 2 | 2 | 4 | 4 | 3 | 6 | 2 | 2 | 6 | 7 | 4 | 6 | 3 | 4 |
| 3 | 6 | 6 | 3 | 6 | 2 | 3 | 3 | 4 | 2 | 6 | 2 | 3 | 4 | 7 | 5 | 5 | 3 | 5 |
| 4 | 5 | 5 | 3 | 5 | 1 | 2 | 3 | 4 | 4 | 6 | 2 | 2 | 4 | 4 | 5 | 5 | 4 | 5 |
| 5 | 5 | 5 | 4 | 5 | 1 | 2 | 3 | 4 | 6 | 6 | 3 | 3 | 3 | 5 | 5 | 5 | 5 | 5 |
| 6 | 4 | 5 | 3 | 4 | 2 | 3 | 3 | 3 | 2 | 2 | 2 | 2 | 4 | 6 | 7 | 7 | 3 | 5 |
| 7 | 5 | 5 | 1 | 3 | 2 | 3 | 4 | 4 | 3 | 6 | 2 | 3 | 3 | 4 | 4 | 6 | 3 | 6 |
| 8 | 3 | 6 | 3 | 5 | 1 | 2 | 3 | 3 | 4 | 6 | 2 | 2 | 3 | 6 | 3 | 8 | 3 | 3 |
| 9 | 3 | 4 | 3 | 4 | 1 | 2 | 3 | 4 | 1 | 2 | 2 | 2 | 4 | 6 | 6 | 7 | 6 | 6 |
| 10 | 5 | 7 | 3 | 6 | 2 | 4 | 3 | 4 | 2 | 7 | 2 | 3 | 4 | 6 | 6 | 6 | 4 | 6 |
| 11 | 3 | 5 | 3 | 4 | 1 | 2 | 4 | 5 | 3 | 5 | 2 | 2 | 4 | 4 | 6 | 7 | 4 | 5 |
| 12 | 3 | 5 | 2 | 4 | 1 | 2 | 3 | 3 | 1 | 2 | 2 | 2 | 3 | 6 | 4 | 6 | 4 | 6 |
| 13 | 6 | 6 | 3 | 4 | 2 | 2 | 4 | 4 | 1 | 1 | 1 | 3 | 6 | 6 | 6 | 7 | 4 | 7 |
| 14 | 4 | 4 | 4 | 6 | 2 | 2 | 3 | 5 | 3 | 7 | 2 | 2 | 4 | 6 | 6 | 8 | 4 | 6 |
| 15 | 5 | 5 | 3 | 3 | 2 | 2 | 3 | 3 | 2 | 4 | 2 | 5 | 4 | 6 | [symbol] | 7 | 4 | 6 |
| 16 | 6 | 7 | 2 | 4 | 1 | 3 | 4 | 5 | 2 | 7 | 2 | 6 | 5 | 6 | 4 | 7 | 6 | 5 |
| 17 | 1 | 6 | 3 | 4 | 2 | 2 | 3 | 3 | 6 | 6 | 1 | 2 | 4 | 6 | 6 | 6 | 3 | 4 |
| 18 | 5 | 6 | 4 | 4 | 1 | 3 | 4 | 4 | 1 | 5 | 2 | 2 | 4 | 6 | 4 | 6 | 3 | 2 |
| 19 | 5 | 5 | 4 | 4 | 1 | 1 | 3 | 4 | 6 | 6 | 2 | 4 | 3 | 6 | 5 | 5 | 3 | 6 |
| 20 | 5 | 6 | 3 | 3 | 1 | 2 | 4 | 5 | 2 | 6 | 6 | 5 | 6 | 6 | 6 | 6 | 4 | 5 |
| 21 | 6 | 6 | 3 | 5 | 1 | 2 | 4 | 4 | 6 | 6 | 2 | 2 | 6 | 6 | 4 | 6 | 2 | 4 |
| 22 | 5 | 5 | 4 | 4 | 2 | 2 | 3 | 4 | 2 | 6 | 2 | 3 | 6 | 6 | 4 | 4 | 4 | 4 |
| 23 | 3 | 5 | [symbol] | 6 | 2 | 2 | 3 | 4 | 2 | 2 | 2 | 2 | 3 | 6 | 4 | 5 | 3 | 4 |
| 24 | 4 | 5 | 2 | 4 | 2 | 2 | 3 | 4 | 2 | 4 | 2 | 3 | 4 | 6 | 4 | 4 | 3 | [symbol] |
| 25 | 4 | 4 | [symbol] | 5 | 3 | 3 | 4 | 4 | 1 | 6 | 2 | 2 | 4 | 5 | 4 | [symbol] | 3 | 5 |
| 26 | 6 | 6 | 3 | 3 | 1 | 3 | 4 | 4 | 6 | 6 | 2 | 5 | 4 | 6 | 4 | 6 | 3 | 4 |
| 27 | 1 | [symbol] | 4 | 6 | 2 | 2 | 4 | 6 | 2 | 5 | 2 | 2 | 6 | 6 | 4 | 5 | 6 | 6 |
| 28 | 5 | 6 | 3 | 6 | 1 | 2 | 3 | 3 | 1 | 7 | 2 | 2 | 4 | [symbol] | 4 | 6 | 4 | 5 |
| 29 | 1 | [symbol] | 3 | 3 | 1 | 4 | 4 | 4 | 1 | 7 | 2 | 3 | 3 | 4 | 4 | 7 | 4 | 5 |
| 30 | 4 | 4 | 4 | 4 | 2 | 3 | 3 | 3 | 1 | 1 | 2 | 4 | 5 | 6 | 4 | 6 | 5 | 5 |
| 31 | 6 | 6 | 4 | 4 | 2 | 4 | 3 | 4 | 2 | 6 | 2 | 4 | 6 | 6 | 4 | 6 | 4 | 4 |
| 32 | 5 | 5 | 3 | 4 | 1 | 2 | 4 | 4 | 1 | 7 | 3 | 3 | 4 | 6 | 6 | 6 | 3 | 4 |
| 33 | 5 | 6 | 3 | 4 | 2 | 3 | 3 | 4 | 6 | 8 | 4 | [symbol] | 6 | 6 | 4 | 6 | 3 | 4 |
| 34 | 3 | 5 | 4 | 5 | 3 | 3 | 3 | 4 | 3 | 6 | 2 | 2 | 3 | 6 | 4 | 5 | 5 | 6 |
| 35 | 5 | 5 | 3 | 3 | 1 | 2 | 4 | 5 | 3 | 6 | 2 | 3 | 6 | 6 | 1 | 7 | 2 | 4 |
| 36 | 6 | 6 | 3 | 3 | 1 | 3 | 3 | 3 | 2 | 6 | 2 | 2 | 6 | 6 | 3 | 7 | 3 | 4 |
| 37 | 5 | 5 | 3 | 4 | 2 | 3 | 2 | 4 | 6 | 6 | 2 | 3 | 4 | 4 | 4 | 4 | 5 | 5 |
| 38 | 5 | 6 | 3 | 4 | 1 | 2 | 3 | 5 | 6 | 6 | 2 | 3 | 6 | 6 | 6 | 6 | 3 | 5 |
| 39 | 6 | 6 | 6 | 6 | 2 | 3 | 4 | 4 | 2 | 6 | 2 | 2 | 3 | 6 | 5 | 5 | 4 | 5 |
| 40 | 4 | 4 | 3 | 4 | 2 | 2 | 4 | 5 | 2 | 2 | 2 | 3 | 4 | 6 | 6 | 7 | [symbol] | 5 |
| 41 | 1 | 5 | 2 | 4 | 3 | 3 | 3 | 4 | 2 | 3 | 2 | 2 | 6 | 6 | 6 | 6 | 4 | 5 |
| 42 | 5 | 6 | 1 | 5 | 2 | 2 | 3 | 3 | 1 | 2 | 2 | 2 | 6 | 6 | 4 | 4 | 4 | 4 |
| 43 | 4 | 5 | 2 | 3 | 2 | 3 | 1 | 3 | 3 | 3 | 2 | 2 | 4 | 4 | 7 | 8 | 4 | 6 |
| 44 | 1 | 5 | 3 | 5 | 2 | 2 | 4 | 4 | 1 | 6 | 2 | 3 | 6 | 6 | 7 | 8 | 3 | 5 |
| 45 | 2 | 2 | 4 | 4 | 2 | 2 | 3 | 4 | 1 | 3 | 2 | 2 | 3 | 4 | 4 | 6 | 4 | 5 |
| 46 | 5 | [symbol] | 3 | 3 | 2 | 2 | 4 | 4 | 2 | 2 | 4 | 4 | 4 | 4 | 5 | 7 | 5 | 6 |
| 47 | 3 | 3 | 1 | 1 | 2 | 3 | 3 | 4 | 2 | 6 | 3 | 5 | 2 | 6 | 4 | 6 | 4 | 5 |
| 48 | 6 | 6 | 1 | 4 | 1 | 2 | 3 | 6 | 2 | 3 | 2 | 2 | 4 | 6 | 7 | 7 | 4 | 4 |
| 49 | 8 | 6 | 3 | 3 | 1 | 2 | 4 | 5 | 1 | 6 | 2 | 2 | 3 | 6 | 4 | 6 | 3 | 5 |
| 50 | 5 | 5 | 4 | [symbol] | 2 | 3 | 3 | 5 | 2 | 6 | 2 | 3 | 6 | 6 | 7 | 8 | 4 | 4 |
| 51 | 1 | 5 | 3 | 5 | 1 | 3 | 3 | 3 | 6 | 6 | 2 | 2 | 4 | 6 | 6 | 7 | 5 | 6 |
| 52 | 3 | 6 | 3 | 6 | 2 | 2 | 4 | 4 | 1 | 6 | 2 | 3 | 5 | 6 | 7 | 8 | 4 | 5 |
| 53 | [symbol] | 6 | [symbol] | 1 | 2 | 2 | 3 | 3 | 4 | [symbol] | 6 | 5 | 3 | 5 | 4 | 7 | 4 | 5 |
| 54 | 5 | [symbol] | 3 | 3 | 1 | 3 | 4 | 4 | 2 | 2 | 2 | 2 | 6 | 7 | 4 | 6 | 4 | 4 |
| 55 | 6 | 6 | 3 | 3 | 2 | 3 | 3 | 3 | 1 | 1 | 2 | 4 | 4 | 6 | 4 | 4 | 4 | 6 |
| 56 | 4 | 5 | 3 | 4 | 2 | 2 | 3 | 3 | 1 | 1 | 2 | 2 | 4 | 4 | 7 | 7 | 5 | 5 |
| 57 | 4 | 4 | 4 | 6 | 2 | 2 | 2 | 4 | 1 | 2 | 3 | 4 | 3 | 4 | 5 | 7 | 3 | 4 |
| 58 | 5 | 5 | 4 | 6 | 2 | 2 | 4 | 6 | 6 | 6 | 2 | 4 | 4 | 6 | 5 | 7 | 6 | 6 |
| 59 | 5 | 5 | 3 | 4 | 2 | 4 | 3 | 4 | 1 | 2 | 3 | 6 | 2 | 3 | 6 | 6 | 3 | 5 |

Presuming the No 1 of the Table 2 and 3 as grandmother (suspected-grandmother), the possibility of granddaughter-cannot-be was analyzed for all the rest people. Again, presuming the No 2 of the Table 2 and 3 as grandmother, the possibility of granddaughter-can-be was analyzed for all the rest people. Precisely, if any STR that is not accord with alleles of suspected-grandmother is included among 18 STRs of suspected-granddaughter, there is no chance of blood-relation between them. Meanwhile, if every alleles of suspected-granddaughter are shared on grandmother's alleles, there is high chance of blood-relation between them. The present inventors performed the same test as above with DNA profiles of 59 people. As a result, 1711 pairs could be analyzed and there was just one case that suspected-granddaughter was proved to be real granddaughter.

Therefore, the chance that suspected-granddaughter's STR alleles are accidentally consistent with those of suspected-grandmother, so that she is regarded as a biological daughter of deceased father is probably 1/1711, which means that the method for determining blood-relationship by typing STR alleles on X chromosome of the present invention is trustworthy.

The present invention also provides a DNA typing kit that could be effectively used for determining blood-relationship between suspected-father and suspected-daughter.

DNA typing kit of the present invention includes a container of primers enabling to amplify X-STR. In addition, the kit also includes molecular weight marker that consists of standard alleles DNA of each X-STR. Factors that are used for providing proper conditions for PCR amplification such as buffer solution, amplification enzyme, nucleotides, etc, can be further included. The preferable STR for the present invention is the one that locates on X chromosome and is distributed evenly. One or more STRs selected from a group consisting of DXS7133, DXS9895, DXS9898, DXS6807, DXS6803, DXS8378, DXS7132, DXS6789, GATA31E08, DXS9900, GATA144D04, GATA165B12, GATA186D06, GATA164A09, DXS6806, ATA28C05, DXS6804 and DXS6797 could be used. Primers that are able to amplify STRs used for test selectively are used for the DNA typing kit of the present invention and especially, primers shown in Table 4 can be preferably used.

TABLE 4
Base sequences of primers and STRs of X chromosome

| | Locus | Direction | Primer Sequence |
|---|---|---|---|
| 1 | DXS7133 | F | SEQ. ID. No 1 |
| | | R | SEQ. ID. No 2 |
| 2 | DXS9895 | F | SEQ. ID. No 3 |
| | | R | SEQ. ID. No 4 |
| 3 | DXS9898 | F | SEQ. ID. No 5 |
| | | R | SEQ. ID. No 6 |
| 4 | DXS6807 | F | SEQ. ID. No 7 |
| | | R | SEQ. ID. No 8 |
| 5 | DXS6803 | F | SEQ. ID. No 9 |
| | | R | SEQ. ID. No 10 |
| 6 | DXS8378 | F | SEQ. ID. No 11 |
| | | R | SEQ. ID. No 12 |
| 7 | DXS7132 | F | SEQ. ID. No 13 |
| | | R | SEQ. ID. No 14 |
| 8 | DXS6789 | F | SEQ. ID. No 15 |
| | | R | SEQ. ID. No 16 |
| 9 | GATA31E08 | F | SEQ. ID. No 17 |
| | | R | SEQ. ID. No 18 |
| 10 | DXS9900 | F | SEQ. ID. No 19 |
| | | R | SEQ. ID. No 20 |
| 11 | GATA144D04 | F | SEQ. ID. No 21 |
| | | R | SEQ. ID. No 22 |
| 12 | GATA165B12 | F | SEQ. ID. No 23 |
| | | R | SEQ. ID. No 24 |
| 13 | GATA186D06 | F | SEQ. ID. No 25 |
| | | R | SEQ. ID. No 26 |
| 14 | GATA164A09 | F | SEQ. ID. No 27 |
| | | R | SEQ. ID. No 28 |
| 15 | DXS6806 | F | SEQ. ID. No 29 |
| | | R | SEQ. ID. No 30 |
| 16 | ATA28C05 | F | SEQ. ID. No 31 |
| | | R | SEQ. ID. No 32 |
| 17 | DXS6804 | F | SEQ. ID. No 33 |
| | | R | SEQ. ID. No 34 |
| 18 | DXS6797 | F | SEQ. ID. No 35 |
| | | R | SEQ. ID. No 36 |

Standard alleles DNA used as a molecular weight marker could be obtained by amplifying genome DNA having each alleles through PCR, by cutting cloned alleles with restriction enzymes or by synthesizing artificial nucleic acid.

DNA typing kit of the present invention can also include STR primer attached with signaling material selected from a group consisting of dioxygenin, biotin, radioisotope, fluorescent material, enzyme, and antibody. The examples of the above enzyme are peroxidase, alkaline phosphatase, luciferase, etc, and FITC and TRITC can be used as a fluorescent material. As a coupler, 4-chloro-1-naphtol (4CN), diaminobenzidine (DAB), aminoethyl carbazole (AEC), 2, 2′-azino-bis (3-ethylbenzothiazoline-6-sulfonic acid) (ABTS), o-phenylenediamine (OPD) or tetramethyl benzidine (TMB) can be used.

The above X-STR alleles can be provided as the form of DNA sequence fixed on a supporting material. Precisely, the present inventors had 5′ end or 3′ end of X-STR alleles DNA fixed using avidin-biotin conjugation or antigen-antibody binding, or had DNA chain fixed on the surface containing amine group.

In order to prepare DNA typing membrane or chip, every STR alleles that are the targets for typing, are amplified by PCR and denaturalized, from which single stranded STR alleles are obtained and then fixed onto the supporting material. The primer used for the amplification of STR alleles with PCR has specific material or functional group attached on the 5′ end of a forward primer or a backward primer. At this time, biotin, primary amine, digoxigenin or fluorescent material can be used as specific material or functional group. PCR was repeatedly performed 25-40 times using a primer having specific material or functional group attached thereon and a normal primer to amplify STR alleles up to 10⁵-10⁷ fold. Such amplified STR alleles were heat-denaturalized to obtain single stranded STR alleles. At last, the single stranded STR alleles attached on DNA typing membrane or chip was prepared by reacting specific material or functional group attached on single stranded STR alleles selected among every obtained single stranded STR alleles and supporting material at 60° C. for 1 hour.

The supporting material of DNA typing membrane or chip on which STR is fixed includes nylon membrane, nitrocellulos membrane, glass slide, polycarbonate or synthetic resins, etc. And, avidin, streptavidin, aldehyde group, etc, can be spread on the surface of the supporting material.

As for membrane is used as a supporting material, biotin of STR alleles is combined with avidin or streptoavidin attached on the membrane, so that STR alleles are fixed on the membrane, resulting in the preparation of DNA typing membrane. As for glass slide is used as a supporting material, biotin of STR alleles is combined with avidin or streptoavidin attached on glass slide or primary amine of STR alleles is combined with aldehyde group attached on glass slide, resulting in the preparation of DNA typing chip.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Method for Confirming Real Child by Comparing X-STRs of Grandmother and Granddaughter in the Absence of the Father's DNA

In order to confirm a blood-relationship by comparing X-STRs of grandmother and granddaughter in the absence of father's DNA, the present inventors have selected following STRs from US GenBank; DXS7133, DXS9895, DXS9898, DXS6807, DXS6803, DXS8378, DXS7132, DXS6789, GATA31E08, DXS9900, GATA144D04, GATA165B12, GATA186D06, GATA164A09, DXS6806, ATA28C05, DXS6804 and DXS6797. The above STRs are located on X chromosome, have high rate of heteroconjugation, and are distributed evenly on X chromosome.

Next, the present inventors have investigated the frequencies of alleles of 200 Korean women to estimate the kind and the frequency of the above 18 STR alleles. 5′ end of only one sides of 18 primer pairs each represented by SEQ. ID. No 1 through No 36 was marked with fluorescent material (6-FAM). These primers were used for the amplification of STR alleles. The sizes and the numbers of amplified alleles were shown in Table 5.

TABLE 5
Sizes of PCR-amplified fragments of STR alleles used in
the present invention

| No | DXS7133 | DXS9895 | DXS9898 | DXS6807 | DXS6803 | DXS8378 | DXS7132 | DXS6789 | GATA31E08 |
|---|---|---|---|---|---|---|---|---|---|
| 1 | 100 | 141 | 180 | 249 | 110 | 200 | 276 | 113 | 227 |
| 2 | 104 | 145 | 184 | 253 | 114 | 204 | 280 | 117 | 231 |
| 3 | 108 | 149 | 188 | 257 | 118 | 208 | 284 | 121 | 235 |
| 4 | 112 | 154 | 192 | 261 | 122 | 212 | 288 | 125 | 239 |
| 5 | 116 | 158 | 196 | 265 | 126 | 216 | 292 | 129 | 243 |
| 6 | 120 | | 200 | 269 | 130 | | 296 | 133 | 247 |
| 7 | 124 | | 204 | | 102 | | 300 | 137 | |
| 8 | | | 208 | | 106 | | | 141 | |
| 9 | | | 212 | | | | | 145 | |
| 10 | | | | | | | | 149 | |
| 11 | | | | | | | | 153 | |
| No | DXS9900 | GATA144D04 | GATA165B12 | GATA186D06 | GATA164A09 | DXS6806 | ATA28C05 | DXS6804 | DXS6797 |
| 1 | 108 | 230 | 125 | 192 | 229 | 163 | 273 | 166 | 243 |
| 2 | 112 | 234 | 129 | 200 | 233 | 167 | 276 | 170 | 251 |
| 3 | 116 | 238 | 133 | 204 | 237 | 171 | 279 | 174 | 259 |
| 4 | 120 | 242 | 137 | 208 | 241 | 175 | 282 | 178 | 263 |
| 5 | 124 | 246 | 141 | 212 | 245 | 179 | 285 | 182 | 267 |
| 6 | 128 | 250 | | 216 | 249 | 183 | 288 | 186 | 271 |
| 7 | 132 | 254 | | 220 | 253 | | 291 | 190 | 275 |
| 8 | | | | | 257 | | 294 | 194 | 279 |

<1-1>DNA Extraction

In order to compare X-STR of grandmother with that of granddaughter, the present inventors have extracted DNA from blood of grandmother, granddaughter and granddaughter's mother. Particularly, 500 μl of blood was transmitted into a test tube containing EDTA, a kind of anticoagulant. The test tube was centrifuged for 1 minute and the supernatants were discarded. Added 900 μl of ACE solution (NH₄Cl 8 g, Na₂EDTA 1 g, KH₂PO₄ 0.1 g, pH 7.0/liter) thereto, vortexed thereof for 15 seconds, and mixed thereof by stirrer at room temperature at 30 rpm for 10 minutes. Centrifuged thereof for 1 minute, and the supernatants were discarded. The pellets were resuspended in 300 μl of nuclei lysis buffer (10 mM Tris-HCl, pH 8.0, 400 mM NaCl, 2 mM EDTA). Added 20 μl of 10% SDS and 6 μl of protease K (20 μg/μl) thereto and mixed thereof well. Cultured thereof at 56° C. for 2 hours. Added 100 μl of saturated NaCl into the tube, vortexed thereof for 15 minutes, and left thereof at room temperature for 5 minutes. Centrifuged for 2 minutes and transferred the supernatants into a new test tube. Added two volumes of alcohol into the tube. After closing the tube, shaken the tube up and down slowly about ten times. Transferred floating nucleic acid clot formed in the above test tube into a new test tube containing 500 μl of TE buffer (10 mM Tris-HCl, pH8.0, 1 mM EDTA). Finally, extracted DNA by leaving thereof at 56° C. water bath for overnight. Extracted DNA was quantified with DynaQuant (Hoefer) using fluorescent assay.

<1-2>Multiplexing Amplification of X-STR

In order to amplify STR of DNA extracted in the above Example <1-1>, multiplexing PCR was performed using primer pairs represented by SEQ. ID. No 1-36. STRs were sorted 3 multiplex panels (the first panel: DXS7133, DXS9895, DXS9898, DXS6807, GATA165B12 and DXS6797; the second panel: DXS6803, DXS6806, DXS8378, GATA144D04, DXS7132, DXS6789, GATA186D06, GATA31E08 and ATA28C05; the third panel: GATA172D05, DXS6804 and GATA164A09), and then PCR was performed.

Particularly, reaction buffer was made with 2 ng of DNA extracted in the above Example <1-1>, 10 mM Tris-HCl (pH 8.3), 50 mM KCl, dNTP 200 μM, 0.2 μM of each primer (DXS8378 and ATA28C05: 0.3 μM), 1.5 mM of MgCl₂ and 2.5 U of DNA polymerase, and final volume of the reaction buffer was adjusted to 25 μl with distilled water. Amplification was performed by 30 cycles as follows: a denaturing step at 94° C. for 1 minute, a primer annealing step at 56° C. for 1 minute and an extension step at 72° C. 1 minute. In case of need, the PCR condition was modified through additional experiments.

<1-3> Physical and Chemical Characteristics of Amplified STR

DNA amplified in the above Example <1-2>was analyzed with ABI310, an automatic electrophoresis device, and the results were shown in Table 6.

TABLE 6
STR profiles of grandmother, granddaughter and
granddaughter's mother

| | Size of PCR Fragment | | | Gene Profile | | | | | |
|---|---|---|---|---|---|---|---|---|---|
| STR | Grandmother | Granddaughter | Mother | Grandmother | | Granddaughter | | Mother | |
| DXS | 114 | 110/ | 110/ | 5 | 5 | 4 | 5 | 4 | 6 |
| 7133 | | 114 | 118 | | | | | | |
| DXS | 140 | 140 | 140 | 1 | 1 | 1 | 1 | 1 | 1 |
| 9895 | | | | | | | | | |
| DXS | 201/ | 201/ | 201 | 6 | 7 | 6 | 7 | 6 | 6 |
| 9898 | 205 | 205 | | | | | | | |
| DXS | 252/ | 252/ | 252/ | 2 | 5 | 2 | 5 | 2 | 5 |
| 6807 | 264 | 264 | 264 | | | | | | |
| DXS | 114 | 114/ | 114/ | 2 | 2 | 2 | 3 | 2 | 3 |
| 6803 | | 118 | 118 | | | | | | |
| DXS | 210/ | 206/ | 206/ | 3 | 4 | 2 | 4 | 2 | 3 |
| 8378 | 214 | 214 | 210 | | | | | | |
| DXS | 283/ | 287 | 287 | 3 | 4 | 4 | 4 | 4 | 4 |
| 7132 | 287 | | | | | | | | |
| DXS | 119/ | 136 | 136 | 3 | 7 | 7 | 7 | 7 | 7 |
| 6789 | 136 | | | | | | | | |
| GATA3 | 243 | 239/ | 239 | 5 | 5 | 4 | 5 | 4 | 4 |
| 1E08 | | 243 | | | | | | | |
| DXS | 122/ | 122/ | 105/ | 4 | 5 | 4 | 5 | 1 | 4 |
| 9900 | 126 | 126 | 122 | | | | | | |
| GATA1 | 237 | 237/ | 245/ | 3 | 3 | 3 | 6 | 5 | 6 |
| 44D04 | | 249 | 249 | | | | | | |
| GATA1 | 125/ | 125/ | 125/ | 1 | 3 | 1 | 2 | 1 | 2 |
| 65B12 | 134 | 129 | 129 | | | | | | |
| GATA1 | 205/ | 200/ | 200/ | 3 | 4 | 2 | 4 | 2 | 3 |
| 86D06 | 209 | 208 | 204 | | | | | | |
| GATA1 | 246/ | 250 | 230/ | 5 | 6 | 6 | 6 | 1 | 6 |
| 64A09 | 250 | | 250 | | | | | | |
| DXS | 167/ | 167 | 167/ | 2 | 3 | 2 | 2 | 2 | 3 |
| 6806 | 171 | | 171 | | | | | | |
| ATA | 279/ | 279/ | 282/ | 3 | 6 | 3 | 4 | 4 | 5 |
| 28C05 | 288 | 282 | 285 | | | | | | |
| DXS | 187 | 178/ | 178 | 6 | 6 | 4 | 6 | 4 | 4 |
| 6804 | | 187 | | | | | | | |
| DXS | 259 | 259/ | 259/ | 2 | 2 | 2 | 3 | 2 | 3 |
| 6797 | | 263 | 263 | | | | | | |

<1-4>Comparison of STR Types

Table 6 is showing 18 X-STR gene profiles of granddaughter, grandmother and mother. When mother's genotypes are excluded from granddaughter's, the alleles indicated in dark letters in Table 6 are left. When the two alleles are identical, both alleles are indicated in same dark letters. In order to determine blood-relationship between grandmother and granddaughter, the alleles indicated in dark letters, in other words alleles of the paternal line, seen in granddaughter's gene profile should be included in grandmother's gene profile. As seen in Table 6, alleles of the paternal line were all found in grandmother's gene profile, resulting in the confirmation of the fact that granddaughter was a real child of a deceased father.

### Example 2

### Method for Confirming Real Child by Comparing X-STR of Sisters in the Absence of the Father's DNA

<2-1>DNA Extraction

The present inventors have extracted DNA from blood of 3 sisters and their biological mother with the same method as the above Example <1-1>.

<2-2>Multiplexing Amplification of X-STR

X-STR was amplified by the same method as the above Example <1-2>.

<2-3>Physical and Chemical Characteristics of Amplified STR

Physical and chemical characteristics of amplified DNA were analyzed by the same method as the above Example <1-3>, and the results were shown in Table 7.

TABLE 7
X-STR profiles of mother and daughters

| | Gene Profile | | | | | | | |
|---|---|---|---|---|---|---|---|---|
| STR | Daughter 1 | | Daughter 2 | | Daughter 3 | | Mother | |
| DXS7133 | 4 | 5 | 4 | 5 | 4 | 5 | 4 | 6 |
| DXS9895 | 1 | 1 | 1 | 3 | 1 | 3 | 1 | 1 |
| DXS9898 | 6 | 7 | 6 | 7 | 6 | 7 | 6 | 6 |
| DXS6807 | 2 | 5 | 2 | 5 | 2 | 5 | 2 | 5 |
| DXS6803 | 2 | 2 | 2 | 3 | 2 | 3 | 2 | 3 |
| DXS8378 | 3 | 4 | 2 | 4 | 2 | 4 | 2 | 3 |
| DXS7132 | 3 | 4 | 4 | 4 | 4 | 4 | 4 | 4 |
| DXS6789 | 3 | 7 | 7 | 7 | 7 | 7 | 7 | 7 |
| GATA31E08 | 4 | 5 | 4 | 5 | 4 | 5 | 4 | 4 |
| DXS9900 | 4 | 5 | 4 | 5 | 4 | 5 | 1 | 4 |
| GATA144D04 | 3 | 5 | 3 | 6 | 3 | 6 | 5 | 6 |
| GATA165B12 | 1 | 3 | 1 | 2 | 1 | 2 | 1 | 2 |
| GATA186D06 | 3 | 4 | 2 | 4 | 2 | 4 | 2 | 3 |
| ATA164A09 | 5 | 6 | 6 | 6 | 6 | 6 | 1 | 6 |
| DXS6806 | 2 | 3 | 2 | 2 | 2 | 2 | 2 | 3 |
| ATA28C05 | 3 | 4 | 3 | 4 | 3 | 4 | 4 | 5 |
| DXS6804 | 4 | 6 | 4 | 6 | 4 | 6 | 4 | 4 |
| DXS6797 | 2 | 2 | 2 | 3 | 2 | 3 | 2 | 3 |

<2-4>Comparison of STR Types

Table 7 is showing 18 X-STR gene profiles of three sisters and mother. When mother's genotypes are excluded from sisters', the alleles indicated in dark letters in Table 7 are left. When the two alleles are identical, both alleles are indicated in same dark letters. In order to share the same biological father, the alleles indicated in dark letters, in other words alleles of the paternal line, should be identical. As seen in Table 7, alleles of the paternal line on 18 STR of daughter 2 and daughter 3 were all identical, meaning they shared the same biological parents. It was also confirmed that daughter 1 and daughter 2 or daughter 1 and daughter 3 had different biological fathers when they were sharing the same biological mother.

### INDUSTRIAL APPLICABILITY

As described hereinbefore, the present invention provides a novel method to confirm a blood-relationship that was difficult to be determined with conventional genetic identification. Precisely, the method of the present invention can be effectively used to confirm a blood-relationship between suspected-father and suspected daughter by comparing X-STR types of grandmother and granddaughter or to confirm whether the suspected-sisters have a same biological father in the absence of father's DNA. With the method of the present invention, a blood-relationship can be determined easily and fast using the DNA typing kit of the present invention even in the absence of father's DNA.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for determining blood-relationship by typing STR alleles on X chromosome of grandmother and granddaughter, or sisters in the absence of father's DNA comprising the following steps:
1) extracting DNA from specimens of examinee;
2) obtaining massive STR DNA through PCR performed with STR on X chromosome using the DNA obtained in the above step 1 as a template;
3) investigating the physical and chemical characteristics of STR DNA obtained in the above step 2; and
4) determining blood-relationship by comparing types of STR obtained in the above step 3.

2. The method for determining blood-relationship as set forth in claim 1, wherein one or more specimens of step 1 can be selected from a group consisting of blood, hair, saliva, epidermis, sperm, samples extracted from vagina, separated cells, tissue samples, dandruffs, ashes, etc, mixed, and used.

3. The method for determining blood-relationship as set forth in claim 1, wherein the STR of step 2 can be selected partly or altogether from a group consisting of DXS7133, DXS9895, DXS9898, DXS6807, GATA165B12, DXS6797, DXS6803, DXS6806, DXS8378, GATA144D04, DXS7132, DXS6789, GATA186D06, GATA31E08, ATA28C05, GATA172D05, DXS6804, DXS9900 and GATA164A09.

4. The method for determining blood-relationship as set forth in claim 3, wherein the each STR is amplified by using primer sets represented by SEQ. ID. No 1-36.

5. The method for determining blood-relationship as set forth in claim 1, wherein the PCR of step 2 is multiplexing PCR that enables simultaneous amplification of all different sized 3-10 STR alleles as a bundle.

6. The method as set forth in claim 5, wherein the multiplexing PCR is performed with any panel selected from a group consisting of the first panel (DXS7133, DXS9895, DXS9898, DXS6807, GATA165B12 and DXS6797), the second panel (DXS6803, DXS6806, DXS8378, GATA144D04, DXS7132, DXS6789, GATA186D06, GATA31E08 and ATA28C05) and the third panel (GATA172D05, DXS6804 and GATA164A09).

7. The method for determining blood-relationship as set forth in claim 6, wherein the DXS7133, DXS9895, DXS 9898 and DXS6807 of the first panel, DXS6803, DXS6806, DXS8378, GATA144D04 and DXS7132 of the second panel, GATA172D05, DXS6804 and GATA164A09 of the third panel are marked with a certain fluorescent material and GATA165B12 and DXS6797 of the first panel, DXS6789, GATA186D06, GATA31E08 and ATA28C05 are marked with the other fluorescent material.

8. The method for determining blood-relationship as set forth in claim 1, wherein one of the primers used for the PCR amplification of step 2 is marked with anything selected from a group consisting of radioisotope, fluorescent material, digoxigenin and biotin.

9. The method for determining blood-relationship as set forth in claim 8, wherein one or more fluorescent materials can be selected from a group consisting of 5-carboxyfluorescein (5-FAM), 6-FAM, tetrachlorinated analogue of 6-FAM (TET), hexachlorinated analogue of 6-FAM (HEX), 6-carboxytetramethylrhodamine (TAMRA), 6-carboxy-X-rhodamine (ROX), 6-carboxy-4′,5′-dichloro-2′, 7′-dimethoxyfluorescein (JOE), NED (ABI), Texas Red™-X, Oregon Green™ 488 carboxylic acid (Molecular Probes Inc.), Cy-3, Cy-5, Cy-5.5 (Amercham PLC), fluorescein-6-isothiocyanate (FITC) and tetramethylrhodamine-5-isothiocyanate (TRITC), and used.

10. The method for determining blood-relationship as set forth in claim 1, wherein the physical and chemical characteristics of STR are analyzed by the method selected from a group consisting of the procedure of electrophoresis that measures the length of DNA fragments amplified by PCR, method that use mass spectrometer to measure the mass of the amplified fragments, method that determines the base sequence directly and method that uses DNA array.

11. The method for determining blood-relationship as set forth in claim 1, wherein the physical and chemical characteristics of DNA are analyzed by using allele steps of X-STR as a standard.

12. The method for determining blood-relationship as set forth in claim 1, wherein the sisters are sisters by a different mother.

13. The method for determining blood-relationship as set forth in claim 10, wherein the DNA array is provided as the form of DNA sequence of X-STR allele steps fixed on the surface of supporting material.

14. A DNA typing kit for determining blood-relationship comprising primer sets which are able to amplify the above X-STR, a molecular weight marker consisting of STR allele steps, a general molecular weight marker without STR alleles, amplification enzyme, nucleotides and buffer solution.
